(19) United States
(12) Patent Application Publication
(10) Pub. No.: US 2026/0174684 A1
(43) Pub. Date: Jun. 25, 2026

(54) PROBIOTICS FOR USE IN THE TREATMENT OF TUMOR

(71) Applicants: WINCLOVE HOLDING B.V., Amsterdam (NL); INSTITUT ALLERGOSAN PHARMAZEUTISCHE PRODUKTE FORSCHUNGS- UND VERTRIEBS GMBH, Graz (AT)

(72) Inventors: Anita Frauwallner, Graz (AT); Sandra Johanna Maria Ten Bruggencate, Amsterdam (NL); Jacoline Gerritsen, Amsterdam (NL)

(21) Appl. No.: 19/126,998

(22) PCT Filed: Nov. 1, 2023
(86) PCT No.: PCT/EP2023/080485

§ 371 Date: May 2, 2025

(30) Foreign Application Priority Data
Nov. 3, 2022 (EP) 22205404.1

(51) Int. Cl. A61K 9/00; A23L 33/135; A61K 35/00; A61K 35/745

(52) U.S. Cl. CPC A61K 9/0056; A23L 33/135; A61K 9/0053; A61K 35/745; A61K 2035/115

(57) ABSTRACT

A method of treatment and use of probiotic compositions in treatment of weight loss-related conditions in wasting, and cachexia, includes the steps of administering a probiotic composition to a subject deemed in need of such treatment wherein the composition includes at least two bacteria, each preferably selected from a different genus, the genera preferably selected from the group Lactobacillus spp, Enterococcus spp, and Bifidobacterium spp, and a probiotic composition for use in treatment of a subject having a weight loss-related condition wherein the composition includes at least two bacteria, each preferably selected from a different genus, the genera preferably selected from the group Lactobacillus spp, Enterococcus spp, and Bifidobacterium spp.

### FIELD

The invention related to methods of treatment and use of probiotic compositions in treatment of weight loss-related conditions in wasting and cachexia.

### BACKGROUND

Wasting of skeletal muscle occurs in a variety of diseases including diabetes, cancer, Crohn's disease, chronic obstructive pulmonary disease (COPD), disuse, and denervation (Zhou et al., Cytokine Signaling in Skeletal Muscle Wasting, Trends in Endocrinology & Metabolism, 2016, 27; 335-347).

Muscle wasting is a frequently observed condition that contributes to progressive functional impairment, psychologic distress, and overall reduced resilience (Lenk, K.; Schuler, G.; Adams, V. Skeletal muscle wasting in cachexia and sarcopenia: Molecular pathophysiology and impact of exercise training. J. Cachexia Sarcopenia Muscle 2010, 1, 9-21). Normally, the equilibrium between protein synthesis and breakdown is tightly regulated and influenced by external stimuli such as physical activity and protein intake. However, during muscle wasting, this equilibrium shifts toward muscle protein breakdown, which is often driven by inflammation, either disease- or age-induced. These inflammation-related muscle wasting syndromes are often known as cachexia and sarcopenia, respectively. Because chronic inflammatory diseases such as cancer primarily develop in the elderly, sarcopenia and cachexia can also co-occur (Pan et al., Inflammation and sarcopenia: A focus on circulating inflammatory cytokines, Experimental Gerontology, 154, 2021, 111544).

Often misdiagnosed as a condition simply of weight loss, cachexia is actually a highly complex metabolic disorder involving features of anorexia, anaemia, lipolysis, and insulin resistance (Saini A, Al-Shanti N, Stewart C E. Waste management-cytokines, growth factors and cachexia. Cytokine Growth Factor Rev. 2006 December; 17(6):475-86). A significant loss of lean body mass arises from such conditions, resulting in wasting of skeletal muscle. Unlike starvation, the weight loss seen in chronic illnesses arises equally from loss of muscle and of fat. The cachectic state is particularly problematic in cancer, typifying poor prognosis and often lowering responses to chemotherapy and radiation treatment. More than half of cancer patients suffer from cachexia (herein also identified as cancer- or tumour-induced cachexia or cancer- or tumour-associated cachexia), and strikingly, nearly one-third of cancer deaths are related to cachexia rather than the tumour burden. As reviewed by Al-Zoughbi et al. (Tumour macroenvironment and metabolism. Semin Oncol. 2014 April; 41(2):281-95.), cancer-associated cachexia is the most important tumour-associated systemic syndrome of late-stage malignant disease and not only affects the quality of life of patients with various malignancies but is estimated to be the cause of death in 15%-20% of all cancer patients.

Cachexia has been associated with infections, decubitus ulcers, and even death. Multivariate analyses of risk and prognostic factors in community-acquired pneumonia in the elderly have found that age by itself is not a significant factor related to prognosis. Among the significant risk factors, only nutritional status is amenable to medical intervention. Cachexia in the elderly may have profound consequences: medical, cognitive, and psychiatric disorders may diminish self-reliance in activities of daily living, thus reducing quality of life and increasing the frequency of secondary procedures, hospitalizations, and the need for skilled care. Cachexia is often associated with higher-than-normal concentrations of tumour necrosis factor α (TNF-α), interleukin (IL) 1, IL-6, serotonin, and interferon γ. The role of these proinflammatory cytokines has been established in the cachexia seen in cancer. Reduction in the concentrations of these cytokines is associated with weight gain.

Furthermore, with recent advances in cancer therapy, typically patients may live longer and, therefore, it is of utmost importance to improve the quality of life during this time. In this context, treatment options addressing specific and often systemic complications of end-stage malignant neoplastic disease are needed.

The intestinal microbiota is composed of a complex community of 100 trillion intestinal archaeal and bacterial cells of more than 1000 different species. These bacteria have a distinct metabolism resulting in the production of metabolites and microbe associated molecular patterns. Some of these metabolites are secreted in the hosts'stools and are referred to as faecal volatile organic compounds (VOCs). In cases of cancer, bacterial metabolites may influence the patients'nutrient uptake, metabolism, gut motility and barrier function, and systemic inflammatory responses. Consequently, alterations of the intestinal microbial composition associated with increased pro-inflammatory cytokines and a catabolic state have been observed in animal models of neoplastic disease.

Probiotics have become an increasingly popular research focus in the last three decades. Defined as “live microorganisms that, when administered in adequate amounts, confer a health benefit on the host,” probiotics have been found to have a multitude of effects both locally and systemically in the human body. The study of probiotics and cancer cachexia is still in its infancy, but intriguing new evidence has been proposed. Varian et al. recently evaluated the role of L. reuteri as a probiotic in cancer cachexia (Beneficial Bacteria Inhibit Cachexia. Oncotarget 2016, 7, 11803-11816). Using the widely accepted ApcMIN/+ mouse model of colon cancer cachexia, they administered L. reuteri via drinking water. They found that administration of this lactic-acid Gram-positive bacterium was associated with larger gastrocnemius muscle mass and decreased evidence of muscle atrophy. Oral L. reuteri administration was also associated with an increased lifespan, larger thymus, and a decrease in FoxN1 expression, a transcription factor involved in systemic inflammation. Bindels et al. have led the field in probiotic trials in patients with cancer cachexia (Synbiotic Approach Restores Intestinal Homeostasis and Prolongs Survival in Leukaemic Mice with Cachexia. ISME J. 2016, 10, 1456-1470). In the study, they noted a decrease in the levels of L. reuteri and L. johnsonii/gasseri in a leukemia mouse model of cachexia. Upon supplementation with oral L. reuteri, these mice exhibited reduced expression of muscle atrophy markers, particularly in the gastrocnemius and tibialis muscles. They also demonstrated a reduction in systemic inflammatory cytokines (IL-6, monocyte chemoattractant protein-1, IL-4, granulocyte colony-stimulating factor). In additional studies, they continued to elaborate on this evidence by utilizing a symbiotic approach using both a probiotic (L. reuteri) and prebiotic (inulin-type fructans). They evaluated the effect of this combination in both colon cancer (C26) and leukemic (BaF) mouse models of cancer cachexia. They found that administration of this combination was associated with decreased cancer cell proliferation and muscle wasting. Furthermore, mice treated with L. reuteri and inulin-type fructans showed a decreased morbidity and prolonged survival (Restoring Specific Lactobacilli Levels Decreases Inflammation and Muscle Atrophy Markers in an Acute Leukemia Mouse Model. PLoS ONE 2012, 7, e37971). L. reuteri in probiotics, however, is often considered to contain risks on negative outcomes as it tends to produce histamine which may pose harm to individuals overly sensitive to histamine (Straub et al., Z Lebensm Unters Forsch. 1995 July; 201(1):79-82.). As reviewed by Herremans et al. (The Microbiota and Cancer Cachexia; Int. J. Mol. Sci. 2019, 20, 6267;) the study of probiotics continues to proliferate as it holds promise to become a new treatment modality for patients with cancer cachexia.

Genaro et al (Probiotic supplementation attenuates the aggressiveness of chemically induced colorectal tumor in rats. Life Sci. 2019 Nov. 15; 237:116895) evaluate the effect of a probiotic on the aggressiveness of already chemically induced colorectal tumours in rats. Twenty-five male Fisher 344 rats, 250 g, provided with feed and water ad libitum, were randomly divided into 5 groups (5 rats/group): G_{control}, no treatment; G_{Tumor}, tumor induction; G_{Tumor-5FU}, tumor induction, 5-Fluorouracil applied; G_{Tumor+Prob}, induction of the tumor, supplemented with probiotic; G_{Tumor+5-FU+Prob}, tumor induction, 5-Fluorouracil applied, supplemented with probiotic. For tumor induction, in all rats of the non-control groups 20 mg/kg of 1,2-dimethylhydrazine was applied over a period of at around 10 weeks (intraperitoneally over 4 weeks, followed by an interval of 15 days, and then repeated for a further 4 weeks). Fifteen weeks after onset of tumour induction (five weeks after the final dose of the carcinogen), a 10-week treatment was initiated with 5-Fluorouracil (15 mg/kg, intraperitoneally/week) and/or a commercial probiotic (comprising Lactobacillus acidophilus, Lactobacillus paracasei, Bifidobacterium lactis and Bifidobacterium bifidum at a total of 1×10⁹ CFU, daily/gavage). No effects on tumour onset time nor on tumour development time nor on tumour-induced cachexia were implicitly nor explicitly measured by Genaro et al. These researchers foremost concluded that the at around 15 weeks long protocol used for tumour induction was able to develop pre-neoplastic but aberrant crypt foci (ACF) and subsequently determined treatment effects on the developed tumours, finding (see also Table 1 in Genaro et al) that treatment with both said chemotherapy and/or said probiotic resulted in a somewhat lower aggressiveness of neoplastic histopathological alterations, as evidenced by a reduced number of adenocarcinomas when compared to untreated tumour-induced rats, albeit with proportionally increased numbers of tubular adenomas or in situ carcinomas in the treated rats after 25 weeks after tumour induction. Of note, that less aggressive adenomas or in situ carcinomas replace the more aggressive adenocarcinomas without altering the final totals of tumours per 5 rats found again shows that the treatment of Genaro et al does not bear relevance to reducing tumour onset time nor tumour development time but only indicates reduced aggressiveness of said tumours as true-fully observed by Genaro et al in those rats.

WO/2022/15797 relates to a nutraceutical composition consisting of a Bifidobacterium breve, Bifidobacterium bifidum, Lactobacillus kefiri, Lactobacillus plantarum, Lactobacillus salivarius, Lactobacillus reuteri, a prebiotic such as inulin and a transferrin such as lactoferrin, for use in the treatment of chemotherapy-related diseases. No effects on tumour onset time nor significant effects on tumour development time of probiotic over chemotherapy nor effects on tumour-induced cachexia were implicitly nor explicitly measured or reported in WO/2022/15797. Furthermore, it is specifically taught in WO/2022/15797 that said composition purportedly depends for its effects on cachexia only when a strain of Lactobacillus reuteri is present in said composition with a prebiotic such as inulin and a transferrin such as lactoferrin.

WO/2021/138979 is restricted to a probiotic mineral material composite preparation. Probiotic microspheres are obtained by mixing a probiotic culture solution subjected to anaerobic culture with a mineral material suspension and then continuing to culture; the probiotic is selected from Lactobacillus, Bifidobacterium, Enterococcus faecalis or Bacillus subtilis;and mineral materials are selected from montmorillonite, diatomite, kaolin, attapulgite or clay particles. Moreover, disclosed are the probiotic mineral material composite preparation, an application of the mineral materials in preparation of drugs for inhibiting malignant tumor growth, and corresponding drugs for inhibiting malignant tumor growth. Mouse experiments show that the probiotic mineral material composite preparation as well as the mineral materials alone have inhibiting effect on malignant tumors. Nowhere in WO/2021/138979 is disclosed that probiotic compositions perse (without the minerals) have inhibiting effect on malignant tumors, let alone have effects on any aspects of tumour onset, development, or effects on weight-loss, wasting or cachexia. Nowhere in WO/2021/138979 a probiotic mineral material composite preparation is disclosed wherein probiotic mixtures of at least two different species of Lactobacillus, Bifidobacterium, Enterococcus faecalis or Bacillus subtilis are provided.

### THE INVENTION

The invention provides a method of treatment of a weight loss-related condition of a subject, preferably allowing or inducing increased accumulation of white adipose tissue in said subject, preferably a human subject, comprising administering a probiotic composition to a subject deemed in need of such treatment wherein said composition comprises at least two bacteria, each preferably selected from a different genus, said genera preferably selected from the group Lactobacillus spp (preferably from the group Lactobacillus acidophilus, Lactobacillus paracasei, Lactobacillus rhamnosus, Lactobacillus salivarius or Lactobacillus plantarum), Enterococcus spp (preferably from Enterococcus faecalis or Enterococcus faecium), and Bifidobacterium spp (preferably from Bifidobacterium bifidum or Bifidobacterium lacti). From said Lactobacillus group, and to avoid histamine effects as much as possible, it is preferred to select at least one, preferably at least two, more preferably at least three different bacteria, more preferably at least four, most preferably all five selected from the group Lactobacillus acidophilus, Lactobacillus paracasei, Lactobacillus rhamnosus, Lactobacillus salivarius or Lactobacillus plantarum. As compared to selecting Enterococcus faecalis versus selecting Enterococcus faecium, the latter is preferred since E. faecium is deemed to pose a far more reduced risk on carrying virulence factors than E. faecalis does (Elsner et al. Eur J Clin Microbiol Infect Dis. 2000 January; 19(1):39-42). From the Bifidobacterium group, it is preferred to at least select Bifidobacterium lactis, more preferably both Bifidobacterium bifidum and Bifidobacterium lactis. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another and further preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of a weight loss-related condition comprising administering a probiotic composition to a subject deemed in need of such treatment wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a method of treatment of a weight loss-related condition of a subject, preferably allowing increased accumulation of white adipose tissue in said subject, preferably a human subject, comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62, Bifidobacterium bifidum W23, and Bifidobacterium lactis W51.

In a further preferred embodiment, the invention provides a method of treatment of a weight loss-related condition comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a method of treatment of a weight loss-related condition comprising administering a probiotic composition to a subject deemed in need of such treatment wherein said composition comprises at least nine different bacteria, preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, Bifidobacterium lactis W51.

The invention also provides a method of treatment of wasting of a subject, preferably allowing or inducing increased accumulation of white adipose tissue in said subject, preferably a human subject, comprising administering a probiotic composition to a subject deemed in need of such treatment wherein said composition comprises at least two bacteria, each preferably selected from a different genus, said genera preferably selected from the group Lactobacillus spp, (preferably from the group Lactobacillus acidophilus, Lactobacillus paracasei, Lactobacillus rhamnosus, Lactobacillus salivarius or Lactobacillus plantarum), Enterococcus spp (preferably from Enterococcus faecalis or Enterococcus faecium), and Bifidobacterium spp (preferably from Bifidobacterium bifidum or Bifidobacterium lacti). From said Lactobacillus group, and to avoid histamine effects as much as possible, it is preferred to select at least one, preferably at least two, more preferably at least three different bacteria, more preferably at least four, most preferably all five selected from the group Lactobacillus acidophilus, Lactobacillus paracasei, Lactobacillus rhamnosus, Lactobacillus salivarius or Lactobacillus plantarum.As compared to selecting Enterococcus faecalis versus selecting Enterococcus faecium, the latter is preferred since E. faecium is deemed to pose a far more reduced risk on carrying virulence factors than E. faecalis does (Elsner et al. Eur J Clin Microbiol Infect Dis. 2000 January; 19(1):39-42). From the Bifidobacterium group, it is preferred to at least select Bifidobacterium lactis, more preferably both Bifidobacterium bifidum and Bifidobacterium lactis. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of wasting comprising administering a probiotic composition to a subject deemed in need of such treatment wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, Bifidobacterium lactis.

In a further preferred embodiment, the invention provides a method of treatment of wasting comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62, Bifidobacterium bifidum W23, and Bifidobacterium lactis W51.

In a further preferred embodiment, the invention provides a method of treatment of wasting comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactisW51. In a further preferred embodiment, the invention provides a method of treatment of wasting comprising administering a probiotic composition to a subject deemed in need of such treatment wherein said composition comprises at least nine different bacteria preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

The invention also provides a method of treatment of cachexia of a subject, preferably allowing or inducing increased accumulation of white adipose tissue in said subject, preferably a human subject, comprising administering a probiotic composition to a subject deemed in need of such treatment wherein said composition comprises at least two bacteria, each preferably selected from a different genus, said genera preferably selected from the group Lactobacillus spp, (preferably from the group Lactobacillus acidophilus, Lactobacillus paracasei, Lactobacillus rhamnosus, Lactobacillus salivarius or Lactobacillus plantarum), Enterococcus spp (preferably from Enterococcus faecalis or Enterococcus faecium), and Bifidobacterium spp (preferably from Bifidobacterium bifidum or Bifidobacterium lacti). From said Lactobacillus group, and to avoid histamine effects as much as possible, it is preferred to select at least one, preferably at least two, more preferably at least three different bacteria, more preferably at least four, most preferably all five selected from the group Lactobacillus acidophilus, Lactobacillus paracasei, Lactobacillus rhamnosus, Lactobacillus salivarius or Lactobacillus plantarum.As compared to selecting Enterococcus faecalis versus selecting Enterococcus faecium, the latter is preferred since E. faecium is deemed to pose a far more reduced risk on carrying virulence factors than E. faecalis does (Elsner et al. Eur J Clin Microbiol Infect Dis. 2000 January; 19(1):39-42). From the Bifidobacterium group, it is preferred to at least select Bifidobacterium lactis, more preferably both Bifidobacterium bifidum and Bifidobacterium lactis.In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of cachexia comprising administering a probiotic composition to a subject deemed in need of such treatment wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a method of treatment of cachexia comprising administering a probiotic composition to a subject deemed in need of such treatment wherein said composition comprises bacteria, each preferably selected from a different species, said species preferably selected from the group of Lactobacillus acidophilus W55 and/or W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, Bifidobacterium lactis W51.

In a further preferred embodiment, the invention provides a method of treatment of cachexia comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactisW51. In a further preferred embodiment, the invention provides a method of treatment of cachexia comprising administering a probiotic composition to a subject deemed in need of such treatment wherein said composition comprises at least nine different bacteria, preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62, Lactobacillus plantarum W1, Bifidobacterium bifidum W23, and Bifidobacterium lactis W51.

The invention also provides a method of treatment of neoplastic disease comprising administering a probiotic composition to a subject deemed in need of such treatment wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of neoplastic disease comprising administering a probiotic composition to a subject deemed in need of such treatment wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a method of treatment of neoplastic disease comprising administering a probiotic composition to a subject deemed in need of such treatment wherein said composition comprises at least eight bacteria, each preferably selected from a different species, said species preferably selected from the group of Lactobacillus acidophilus W55 and/or W37, Lactobacillus paracasei W20, Lactobacillus rhamnosus W71, Enterococcus faecium W54, Lactobacillus salivarius W24, Lactobacillus plantarum W62 and/or W1, Bifidobacterium bifidum W23, and Bifidobacterium lactis W51.

In a further preferred embodiment, the invention provides a method of treatment of neoplastic disease comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and/or W1, Bifidobacterium bifidum W23, and Bifidobacterium lactis W51.

In a further preferred embodiment, the invention provides a method of treatment of neoplastic disease comprising administering a probiotic composition to a subject deemed in need of such treatment wherein said composition comprises at least nine different bacteria, preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

The invention also provides a method of treatment of malignant neoplastic disease comprising administering a probiotic composition to a subject deemed in need of such treatment wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of malignant neoplastic disease comprising administering a probiotic composition to a subject deemed in need of such treatment wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a method of treatment of malignant neoplastic disease comprising administering a probiotic composition to a subject deemed in need of such treatment wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a method of treatment of malignant neoplastic disease comprising administering a probiotic composition to a subject deemed in need of such treatment wherein said composition comprises at least nine different bacteria, preferably selected from the group of Lactobacillus acidophilus W55 and/or W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

The invention also provides a method of treatment of malignant neoplastic disease comprising administering a probiotic composition to a subject deemed in need of such treatment, wherein said subject may, is or has also been subjected to anti-cancer therapy such as immunotherapy, chemotherapy, radiation therapy and/or surgical therapy, wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of malignant neoplastic disease comprising administering a probiotic composition to a subject deemed in need of such treatment, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a method of treatment of malignant neoplastic disease comprising administering a probiotic composition to a subject deemed in need of such treatment, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a method of treatment of malignant neoplastic disease comprising administering a probiotic composition to a subject deemed in need of such treatment, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and/or W1, Bifidobacterium bifidum W23, and Bifidobacterium lactisW51. In a further preferred embodiment, the invention provides a method of treatment of malignant neoplastic disease comprising administering a probiotic composition to a subject deemed in need of such treatment, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

The invention also provides a method of treatment of malignant neoplastic disease, wherein said treatment is also directed at delaying cancer onset and/or delaying cancer development, comprising administering a probiotic composition to a subject deemed in need of such treatment, wherein said subject may, or may not, is or has also been subjected to anti-cancer therapy such as immunotherapy, chemotherapy, radiation therapy and/or surgical therapy, wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a method of treatment of malignant neoplastic disease, wherein said treatment is also directed at delaying cancer onset and/or delaying cancer development, comprising administering a probiotic composition to a subject deemed in need of such treatment, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a method of treatment of malignant neoplastic disease, wherein said treatment is also directed at delaying cancer onset and/or delaying cancer development, comprising administering a probiotic composition to a subject deemed in need of such treatment, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a method of treatment of malignant neoplastic disease, wherein said treatment is also directed at delaying cancer onset and/or delaying cancer development, comprising administering a probiotic composition to a subject deemed in need of such treatment, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a method of treatment of malignant neoplastic disease, wherein said treatment is also directed at delaying cancer onset and/or delaying cancer development, comprising administering a probiotic composition to a subject deemed in need of such treatment, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

The invention provides a probiotic composition for use in treatment of a subject having a weight loss-related condition wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a weight loss-related condition wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a probiotic composition for use in treatment of a subject having a weight loss-related condition wherein said composition comprises at least eight bacteria, each preferably selected from a different species, said species preferably selected from the group of Lactobacillus acidophilus W55 and/or W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a weight loss-related condition wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a weight loss-related condition wherein said composition comprises at least nine bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, Bifidobacterium lactis W51.

The invention also provides a probiotic composition for use in treatment of a subject having a condition of wasting wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a condition of wasting wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis,.

In a further preferred embodiment, the invention provides a probiotic composition for use in treatment of a subject having a condition of wasting wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a probiotic composition for use in treatment of a subject having a condition of wasting wherein said composition comprises at least eight bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a probiotic composition for use in treatment of a subject having a condition of wasting wherein said composition comprises at least nine bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a probiotic composition for use in treatment of a subject having a condition of wasting wherein said composition comprises ten bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

The invention also provides a probiotic composition for use in treatment of a subject having a condition of cachexia wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a condition of cachexia wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis,

In a further preferred embodiment, the invention provides a probiotic composition for use in treatment of a subject having a condition of cachexia wherein said composition comprises at least nine bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of cachexia wherein said composition comprises ten bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a probiotic composition for use in treatment of a subject having a condition of cachexia wherein said composition comprises at ten bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of cachexia wherein said composition comprises at least eight bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a probiotic composition for use in treatment of a subject having a condition of cachexia wherein said composition comprises at least nine bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In a further preferred embodiment, the invention provides a probiotic composition for use in treatment of a subject having a condition of cachexia wherein said composition comprises ten bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

The invention also provides a probiotic composition for use in treatment of a subject having a condition of neoplastic disease wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp.

In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a condition of neoplastic disease wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

In a further preferred embodiment, the invention provides a probiotic composition for use in treatment of a subject having a condition of neoplastic disease wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of neoplastic disease wherein said composition comprises at least nine bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of neoplastic disease wherein said composition comprises at ten bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

The invention also provides a probiotic composition for use in treatment of a subject having a condition of malignant neoplastic disease wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a condition of malignant neoplastic disease wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis,

In a further preferred embodiment, the invention provides a probiotic composition for use in treatment of a subject having a condition of malignant neoplastic disease wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

The invention also provides a probiotic composition for use in treatment of a subject having a condition of malignant neoplastic disease, wherein said subject may, is or has also been subjected to anti-cancer therapy such as immunotherapy, chemotherapy, radiation therapy and/or surgical therapy, wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a condition of malignant neoplastic disease, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis, In a further preferred embodiment, the invention provides a probiotic composition for use in treatment of a subject having a condition of malignant neoplastic disease, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease, wherein said subject may, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

The invention also provides a probiotic composition for use in treatment of a subject having a condition of malignant neoplastic disease, wherein said treatment is also directed at delaying cancer onset and/or delaying cancer development, wherein said subject may, or may not, is or has also been subjected to anti-cancer therapy such as immunotherapy, chemotherapy, radiation therapy and/or surgical therapy, wherein said composition comprises at least two bacteria, each preferably selected from a different genus, said genera preferably selected from the group Lactobacillus spp, Enterococcus spp, and Bifidobacterium spp. In a preferred embodiment, the composition comprises at least a bacterium selected from Lactobacillus spp, and a bacterium selected from Enterococcus spp. In another preferred embodiment, the composition comprises at least a bacterium selected from Enterococcus spp, and a bacterium selected from Bifidobacterium spp. In another preferred embodiment, the composition comprises at least a bacterium selected from Lactobacillus spp, and a bacterium selected from Enterococcus spp and a bacterium selected from Bifidobacterium spp.

In another preferred embodiment, the invention provides a probiotic composition for use in treatment of a subject having a condition of malignant neoplastic disease, wherein said treatment is also directed at delaying cancer onset and/or delaying cancer development, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis,

In a further preferred embodiment, the invention provides a probiotic composition for use in treatment of a subject having a condition of malignant neoplastic disease, wherein said treatment is also directed at delaying cancer onset and/or delaying cancer development, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease, wherein said treatment is also directed at delaying cancer onset and/or delaying cancer development, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease, wherein said treatment is also directed at delaying cancer onset and/or delaying cancer development, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W62 and W1, Bifidobacterium bifidum W23, and Bifidobacterium lactis W51.

In a further preferred embodiment, the invention provides a probiotic composition for use in treatment of a subject having a condition of malignant neoplastic disease, wherein said treatment is also directed at delaying cancer onset and/or delaying cancer development, wherein said subject may, or may not, is or has also been subjected to anti-cancer therapy such as immunotherapy, chemotherapy, radiation therapy and/or surgical therapy, wherein said composition comprises at least eight bacteria, each preferably selected from a different species, said species preferably selected from the group of Lactobacillus acidophilus W55 and W37, Lactobacillus paracasei W20, Lactobacillus rhamnosus W71, Enterococcus faecium W54, Lactobacillus salivarius W24, Lactobacillus plantarum W1, Bifidobacterium bifidum W23, Bifidobacterium lactis W51.

In yet another preferred embodiment, the invention provides a method of reducing tumour onset time in a subject comprising administering a probiotic composition to a subject deemed in need of such treatment wherein said composition comprises at least two bacteria each selected from a different genus, said genera preferably selected from the group Lactobacillus spp, Enterococcus spp, and Bifidobacterium spp. Preferably, the invention provides a method of reducing tumour onset time in a subject comprising administering a probiotic composition to a subject deemed in need of such treatment wherein said composition comprises bacteria preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

Preferably, said composition for reducing tumor onset time as provided herein comprises at least eight bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51, more preferably, said composition comprises at least nine bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51, most preferably, said composition comprises ten bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51.

In yet another preferred embodiment, the invention provides a method of reducing tumour development time in a subject comprising administering a probiotic composition to a subject deemed in need of such treatment wherein said composition comprises at least two bacteria each selected from a different genus, said genera preferably selected from the group Lactobacillus spp, Enterococcus spp, and Bifidobacterium spp. Preferably, the invention provides a method of reducing tumour development time in a subject comprising administering a probiotic composition to a subject deemed in need of such treatment wherein said composition comprises bacteria preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

Preferably, said composition for reducing tumour development time as provided herein comprises at least eight bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51, more preferably, said composition comprises at least nine bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51, most preferably, said composition comprises ten bacteria species, said species preferably selected from the group of Lactobacillus acidophilus W55 and W37, Lactobacillus paracasei W20, Lactobacillus rhamnosus W71, Enterococcus faecium W54, Lactobacillus salivarius 24, Lactobacillus plantarum W62 and W1, Bifidobacterium bifidum W23, and Bifidobacterium lactis W51. It is most preferred that said method reducing tumour onset time in a subject or reducing tumour development time in a subject is applied in a condition wherein said subject may, is or has also been subjected to anti-cancer therapy such as immunotherapy, chemotherapy, radiation therapy and/or surgical therapy.

Multispecies probiotic product Ecologic AAD is an established multispecies probiotic composition comprising bacterial strains from Lactobacillus, Enterococcus and Bifidobacterium spp and commercially available at Winclove (www.winclove.com; Hulstweg 11, 1032 LB, Amsterdam) and also commercially available at Institut Allergosan (www.allergosan.com; Gmeinstraße 13, 8055 Graz, Österreich). It contains the following generally available probiotic strains: Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis, Neuroblastoma (NB) cell lines are transformed, neural crest derived cells, capable of unlimited proliferation in vitro and vivo. These cell lines retain the ability of differentiation into neuronal cell types on treatment with various agents (Dimitroulakos J, Squire J, Pawlin G, Yeger H. NUB-7: a stable I-type human neuroblastoma cell line inducible along N- and S-type cell lineages. Cell Growth Differ. 1994 April; 5(4):373-84. PMID: 8043511.)

### FIGURE LEGENDS

FIG. 1 The effect of 1.2×10\hat{ }9 CFU probiotic product Ecologic AAD per day for at least 3 weeks on total weight of white adipose tissue (WAT; g/g bodyweight) in athymic Hsd:Fox1nu mice that receive sub-peritoneal implantation of human NB cells (n=12 per group). Animals of the chemotherapy groups receive two intraperitoneal doses 50 mg/kg CTX chemotherapy 8/9 days after the implementation of the human NB cells. p-values <0.05 are considered statistically significant.

FIG. 2 The effect of 1.2×10\hat{ }9 CFU probiotic product Ecologic AAD per day for at least 3 weeks on tumour onset time ((first visibility of tumor as measured in days) in athymic Hsd:Fox1nu mice that receive sub-peritoneal implantation of human NB cells (n=12 per group). p-values <0.05 are considered statistically significant.

FIG. 3 The effect of 1.2×10\hat{ }9 CFU probiotic product Ecologic AAD per day for at least 3 weeks on total tumour development time ((time from sub-peritoneal implantation to euthanasia as measured in days) in athymic Hsd:Fox1nu mice that receive sub-peritoneal implantation of human NB cells (n=12 per group). p-values <0.05 are considered statistically significant.

### DETAILED DESCRIPTION

Cachexia is a multifactorial syndrome defined by weight loss greater than 5%, weight loss greater than 2% in those who have a BMI <20 kg/m2 or depletion in skeletal muscle mass. Muscle wasting is a frequently observed, inflammation-driven condition in aging and disease, known as sarcopenia and cachexia, respectively. Current treatment strategies target the muscle directly and are often not able to reverse the process. Because a reduced gut function is related to systemic inflammation, this might be an indirect target to ameliorate muscle wasting, by administering pro-, pre-, and synbioticsalpha to treat muscle wasting and to elucidate which metabolites and mechanisms affect the organ crosstalk in cachexia. Overall, the study shows that Lactobacillus species pluralis (spp.) and possibly other genera, such as Bifidobacterium, can ameliorate muscle wasting in mouse models. The beneficial effects of Lactobacillus spp. supplementation may be attributed to its potential to improve microbiome balance and to its reported capacity to reduce gut permeability.

Cachexia is seen in approximately 80% of patients with advanced cancer and has been reported to contribute to 30% of cancer deaths (Kalantar-Zadeh, K.; Rhee, C.; Sim, J.; Stenvinkel, P.; Anker, S.; Kovesdy, C. Why Cachexia Kills: Examining the Causality of Poor Outcomes in Wasting Conditions. J. Cachexia Sarcopenia Muscle 2013, 4, 89-94.). The clinical phenomenon of cachexia has been identified for centuries, originally documented by Hippocrates as “the flesh is consumed, the shoulders, clavicles, chest and thighs melt away : : : the illness is fatal”. Modern research has elucidated a complex interplay of cytokines, inflammation, and metabolic derangement. Cachexia includes not only weight loss, but also white adipose tissue (WAT) wasting, muscle atrophy, and decreased appetite, with metabolic dysfunction preceding these physical signs. Cachexia has been noted in many chronic inflammatory conditions such as acquired immunodeficiency syndrome (AIDS), sepsis, autoimmune disorders, chronic lung disease and cancer. Cancer cachexia is particularly devastating as it is predictive of early mortality, poor response to chemotherapy and can even be a direct cause of death. In addition to quantity, cancer cachexia effects the quality of patients'lives. Muscle and adipose wasting along with progressive anorexia can be particularly distressing to patients and their family members. Early interventions such as exercise, nutritional supplementation, counselling, and medications have been trialled to no avail. Several different pharmacologic therapies have been tested with a focus on appetite stimulants, anabolic agents, and metabolic inhibitors. Short courses of corticosteroids, progesterone analogues and more recently cannabinoids have been used to enhance appetite stimulation in patients with cancer cachexia. Anabolic steroids and recombinant growth hormone have been evaluated for their theoretical mechanism of action in muscle anabolism; however, no statistically significant benefit has been discovered. Metabolic inhibitors have also been under investigation for their proposed benefit in decreasing systemic inflammation. Eicosapentaenoic acid (EPA) has been suggested to decrease IL-6, but studies have not consistently shown improvement. Additionally, TNF-alpha_antibodies have not shown any statistically significant benefit in humans despite their mechanism. Overall, pharmaceutical interventions have not shown a significant survival benefit or an improvement in quality of life in patients with cancer cachexia (Bruggeman, A. R.; Kamal, A. H.; LeBlanc, T. W.; Ma, J. D.; Baracos, V. E.; Roeland, E. J. Cancer Cachexia: Beyond Weight Loss. J. Oncol. Pract. 2016, 12, 1163-1171).

Probiotic product Ecologic AAD (van der Geest A M, et al,. Multispecies probiotics promote perceived human health and wellbeing: insights into the value of retrospective studies on user experiences. Benef Microbes. 2021 Oct. 11; 12(5):413-430.) used herein is an established multispecies probiotic composition comprising bacterial strains from Lactobacillus, Enterococcus and Bifidobacterium spp and commercially available at Winclove (www.winclove.com; Hulstweg 11, 1032 LB, Amsterdam) and at Institut Allergosan (www.allergosan.com; Gmeinstraße 13, 8055 Graz, Österreich). It contains the nine following generally available probiotic strains: Lactobacillus acidophilus W55 and Lactobacillus acidophilus W37, Lactobacillus paracasei W20, Lactobacillus rhamnosus W71, Enterococcus faecium W54, Lactobacillus salivarius W24, Lactobacillus plantarum W62 and, Bifidobacterium bifidum W23, Bifidobacterium lactis W51, to which other established probiotic strains, such as preferably Lactobacillus plantarum W1 (=WCFS1) (van den Nieuwboer M et al., Lactobacillus plantarum WCFS1 and its host interaction: a dozen years after the genome. Microb Biotechnol. 2016 July; 9(4):452-65.) may preferably be added for improved performance.

Neuroblastoma (NB) cell lines are transformed, neural crest derived cells, capable of unlimited proliferation in vitro and vivo. These cell lines retain the ability of differentiation into neuronal cell types on treatment with various agents (Dimitroulakos J, Squire J, Pawlin G, Yeger H. NUB-7: a stable I-type human neuroblastoma cell line inducible along N- and S-type cell lineages. Cell Growth Differ. 1994 April; 5(4):373-84. PMID: 8043511).

With an incidence of 10.7 per 1 million, neuroblastoma (NB) represents the most common extracranial malignant tumour in children. NB remains distinct from other solid paediatric tumours due to its biological heterogeneity and range of clinical behaviour spanning from spontaneous regression to cases of highly aggressive metastatic disease unresponsive to standard and investigational anti-cancer treatment.

The intestinal microbiota is composed of a complex community of 100 trillion intestinal archaeal and bacterial cells of more than 1000 different species. These bacteria have a distinct metabolism resulting in the production of metabolites and microbe associated molecular patterns. Some of these metabolites are secreted in the hosts'stools and are referred to as faecal volatile organic compounds (VOCs). In cases of cancer, bacterial metabolites may influence the patients'nutrient uptake, metabolism, gut motility and barrier function, and systemic inflammatory responses. Consequently, alterations of the intestinal microbial composition associated with increased pro-inflammatory cytokines and a catabolic state have been observed in animal models of leukaemia and neuroblastoma.

With modern chemotherapy the 5-year survival of patients with NB has increased to 76%. Amongst others, cyclophosphamide (CTX) belongs to the drugs for SIOPN standard (HRNBL1.7/SIOPEN) chemotherapy of NB. Apart from its impact on tumour growth, chemotherapy may also cause disturbed gut barrier function with marked bacterial translocation and increased inflammation. Additional to the effect on the bowel wall recent studies have also advocated chemotherapy-induced alterations of the intestinal microbiome. Finally, an intact microbiome seems to be essential for the functioning of CTX chemotherapy—for instance by influencing the reprogramming of myeloid cells in the tumour micro-environment.

### Study Design

Cancer therapy is often associated with severe side effects such as drug induced weight loss, also known as chemotherapy-induced cachexia. The aim of this study was to investigate the effects of a multispecies probiotic (OMNI-BIOTIC® 10 AAD) in a chemotherapy mouse model. A total of 24 male BALB/c mice were gavage-fed with the probiotic formulation or water, once a day for 3 weeks. In the third week, the mice received intraperitoneal cyclophosphamide. At euthanasia, the organs were dissected, and serum was sampled for cytokine analysis. Tight junction components, myosin light chain kinase, mucins, and apoptosis markers were detected in the ileum and colon using histological analyses and qRT-PCR. Lipolysis was analyzed by enzymatic activity assay, Western blotting analyses, and qRT-PCR inWAT. The fecal microbiome was measured with 16S-rRNA gene sequencing from stool samples, and fecal volatile organic compounds analysis was performed using gas chromatography/mass spectrometry. The probiotic-fed mice exhibited significantly less body weight loss and adipose tissue wasting associated with a reduced CGI58 mediated lipolysis. They showed significantly fewer pro-inflammatory cytokines and lower gut permeability compared to animals fed without the probiotic. The colons of the probiotic-fed animals showed lower inflammation scores and less goblet cell loss. qRT-PCR revealed no differences in regards to tight junction components, mucins, or apoptosis markers. No differences in microbiome alpha diversity, but differences in beta diversity, were observed between the treatment groups. Taxonomic analysis showed that the probiotic group had a lower relative abundance of Odoribacter and Ruminococcus-UCG014 and a higher abundance of Desulfovibrio. VOC analysis yielded no significant differences. The results of this study indicate that oral administration of the multispecies probiotic OMNi-BIOTIC® 10 AAD could mitigate cyclophosphamide-induced chemotherapy side effects.

Athymic Hsd:Fox1nu mice received subperitoneal implantation of human NB cells (MHH-NB11) (n=12 per group; NB-AAD versus NB-Aqua) as described previously (Obermüller et al, Nutrients. 2020 Jul. 8; 12(7):2029). For CTX chemotherapy, a solution containing 10 mg/ml CTX was prepared by the institution's pharmacy. Animals of the chemotherapy groups received two intraperitoneal doses 50 mg/kg CTX chemotherapy (n=12 per group; CTX-AAD versus CTX-Aqua) 8/9 days after the implementation of the human NB cells.

We included 1.2×10⁹ CFU probiotic product/day by oral gavage in the present study probiotic product comprises bacterial Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

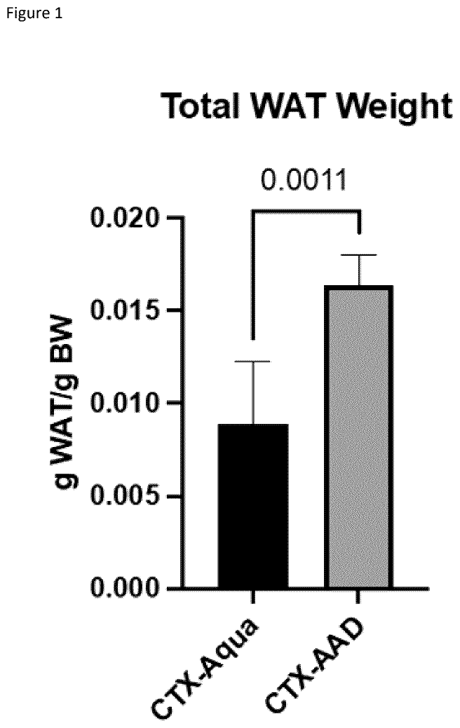

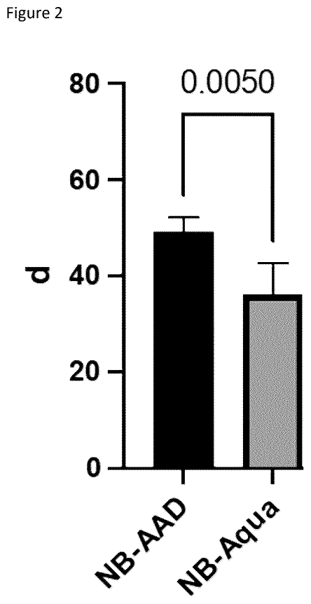

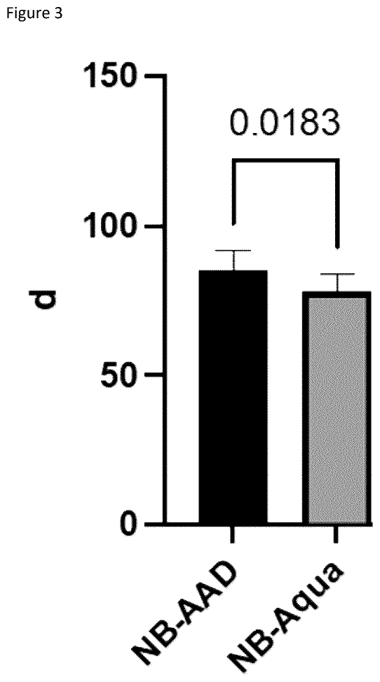

Results are depicted in FIGS. 1, 2 and 3.

### FURTHER EMBODIMENTS

1 A method of treatment of a weight loss-related condition comprising administering a multispecies probiotic composition to a subject deemed in need of such treatment wherein said composition comprises at least two bacteria, each preferably selected from a different genus, said genera preferably selected from the group Lactobacillus spp, Enterococcus spp, and Bifidobacterium spp.
2 A method of treatment of a weight loss-related condition comprising administering a multispecies probiotic composition to a subject deemed in need of such treatment wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.
3 A method of treatment of a weight loss-related condition comprising administering a multispecies probiotic composition to a subject deemed in need of such treatment wherein said composition comprises said species preferably selected from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Bifidobacterium bifidum W23, and Bifidobacterium lactis W51.
4 A method of treatment of a weight loss-related condition comprising administering a multispecies probiotic composition to a subject deemed in need of such treatment wherein said composition comprises said species preferably selected from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Lactobacillus plantarum W1, Bifidobacterium bifidum W23, and Bifidobacterium lactis W51.
5 A method of treatment of a weight loss-related condition comprising administering a multispecies probiotic composition to a subject deemed in need of such treatment wherein said composition comprises said species from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Bifidobacterium bifidum W23, and Bifidobacterium lactis W51.
6 A method of treatment of a weight loss-related condition comprising administering a multispecies probiotic composition to a subject deemed in need of such treatment wherein said composition comprises said species from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Lactobacillus plantarum W1, Bifidobacterium bifidum W23, and Bifidobacterium lactis W51.
7 The method according to anyone of further embodiments 1 to 6 wherein said condition comprises wasting.
8 The method according to anyone of further embodiments 1 to 7 wherein said condition comprises cachexia.
9 The method according to anyone of further embodiments 1 to 8 wherein said condition also comprises a neoplastic disease.
10 The method according to anyone of further embodiments 1 to 9 wherein said condition is a malignant neoplastic disease.
11 The method according to anyone of further embodiments 1 to 10 wherein said subject may, is or has also been subjected to anti-cancer therapy such as immunotherapy, chemotherapy, radiation therapy and/or surgical therapy.
12 The method according to anyone of further embodiments 1 to 11 wherein said treatment is also directed at delaying cancer onset.
13 The method according to anyone of further embodiments 1 to 12 wherein said treatment is also directed at delaying cancer development.
14 The method according to anyone of further embodiments 1 to 13 wherein said condition relates to a solid tumour.
15 A multispecies probiotic composition for use in treatment of a subject having a weight loss-related condition wherein said composition comprises at least two bacteria, each preferably selected from a different genus, said genera preferably selected from the group Lactobacillus spp, Enterococcus spp, and Bifidobacterium spp.
16 A multispecies probiotic composition for use in treatment of a subject having a weight loss-related condition wherein said composition comprises at least eight bacteria, each preferably selected from a different species, said species preferably selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.
17 A multispecies probiotic composition for use in treatment of a subject having a weight loss-related condition wherein said composition comprises species preferably selected from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Bifidobacterium bifidum W23, and Bifidobacterium lactis W51.
18 A multispecies probiotic composition for use in treatment of a subject having a weight loss-related condition wherein said composition comprises species preferably selected from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Lactobacillus plantarum W1, Bifidobacterium bifidum W23, and Bifidobacterium lactis W51.
19 A multispecies probiotic composition for use in treatment of a subject having a weight loss-related condition wherein said composition comprises species from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Bifidobacterium bifidum W23, and Bifidobacterium lactis W51.
20 A multispecies probiotic composition for use in treatment of a subject having a weight loss-related condition wherein said composition comprises species from the group of Lactobacillus acidophilus W37, Lactobacillus acidophilus W55, Lactobacillus paracasei W20, Lactobacillus rhamnosus W71, Enterococcus faecium W54, Lactobacillus salivarius W24, Lactobacillus plantarum W62, Lactobacillus plantarum W1, Bifidobacterium bifidum W23, and Bifidobacterium lactis W51.
21 The probiotic composition according to further embodiments 15 to 20 wherein said condition comprises wasting.
22 A probiotic composition according to anyone of further embodiments 15 to 21 wherein said condition comprises cachexia.
23 A probiotic composition according to anyone of further embodiments 15 to 22 wherein said condition also comprises a neoplastic disease.
24 A probiotic composition according to anyone of further embodiments 15 to 23 wherein said condition is a malignant neoplastic disease.
25 A probiotic composition according to anyone of further embodiments 15 to 24 wherein said subject may, is or has also been subjected to anti-cancer therapy such as immunotherapy, chemotherapy, radiation therapy and/or surgical therapy.
26 A probiotic composition according to anyone of further embodiments 15 to 25 wherein said treatment is also directed at delaying tumour onset.
27 A probiotic composition according to anyone of further embodiments 15 to 26 wherein said treatment is also directed at delaying tumour development.
28 A probiotic composition according to anyone of further embodiments 15 to 27 wherein said condition relates to a solid tumour.

What is claimed is:

1-16. (canceled)

17. A method of one or more of the following:
treatment of a weight loss-related condition of wasting or cachexia,
reducing tumour onset time in a subject,
reducing tumour development time in a subject,
said method comprising the step of administering a probiotic composition to a subject deemed in need of such treatment wherein said composition comprises at least two bacteria each selected from a different genus, said genera selected from the group Lactobacillus spp, Enterococcus spp, and Bifidobacterium spp.

18. The method according to claim 17 wherein said condition also comprises a malignant, neoplastic disease.

19. The method according to claim 17 wherein said subject one of may, is or has been subjected to anti-cancer therapy including at least one of immunotherapy, chemotherapy, radiation therapy and surgical therapy.

20. A method of one or more of the following:
treatment of a weight loss-related condition,
reducing tumour onset time in a subject,
reducing tumour development time in a subject, said method comprising the step of administering a probiotic composition to a subject deemed in need of such treatment wherein said composition comprises bacteria selected from a different species, said species selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

21. The method according to claim 20 wherein said condition also comprises a malignant, neoplastic disease.

22. The method according to claim 20 wherein said subject one of may, is or has been subjected to anti-cancer therapy including at least one of immunotherapy, chemotherapy, radiation therapy and surgical therapy.

23. A probiotic composition for use in one or more of the following:
treatment of a subject having a weight loss-related condition of wasting or cachexia,
a method of reducing tumor onset time in a subject comprising administering a probiotic composition to a subject deemed in need of such treatment
a method of reducing tumor development time in a subject comprising administering a probiotic composition to a subject deemed in need of such treatment,
said probiotic composition comprising at least two bacteria each selected from a different genus, said genera selected from the group Lactobacillus spp, Enterococcus spp, and Bifidobacterium spp.

24. A probiotic composition according to claim 23 wherein said condition also comprises a malignant, neoplastic disease.

25. A probiotic composition according to claim 23 wherein said subject one of may, is or has been subjected to anti-cancer therapy including at least one of immunotherapy, chemotherapy, radiation therapy and surgical therapy.

26. A probiotic composition for use in one or more of the following:
treatment of a subject having a weight loss-related condition of wasting or cachexia,
a method of reducing tumor onset time in a subject comprising administering a probiotic composition to a subject deemed in need of such treatment
a method of reducing tumor development time in a subject comprising administering a probiotic composition to a subject deemed in need of such treatment,
said probiotic composition comprising bacteria each selected from a different species, said species selected from the group of Lactobacillus acidophilus, Lactobacillus paracasei, Lactobacillus rhamnosus, Enterococcus faecium, Lactobacillus salivarius, Lactobacillus plantarum, Bifidobacterium bifidum, and Bifidobacterium lactis.

27. A probiotic composition according to claim 26 wherein said condition also comprises a malignant, neoplastic disease.

28. A probiotic composition according to claim 26 wherein said subject one of may, is or has been subjected to anti-cancer therapy including at least one of immunotherapy, chemotherapy, radiation therapy and surgical therapy.
